(19) United States
(12) Patent Application Publication
(10) Pub. No.: US 2024/0385528 A1
(43) Pub. Date: Nov. 21, 2024

(54) METHODS OF FORMING BIOACTIVE PATTERNS USING BEAM PEN LITHOGRAPY-CONTROLLED CROSS-LINKING PHOTOPOLYMERIZATION

(71) Applicants: NORTHWESTERN UNIVERSITY, Evanston, IL (US); TERRA-Print, LLC, Skokie, IL (US)

(72) Inventors: Chad A. Mirkin, Wilmette, IL (US); Xinpeng Zhang, Evanston, IL (US); Andrey Ivankin, Chicago, IL (US); Shaowei Ding, Des Plaines, IL (US)

(21) Appl. No.: 18/695,856

(22) PCT Filed: Sep. 30, 2022
(86) PCT No.: PCT/US22/45366

§ 371 Date: Mar. 27, 2024

Related U.S. Application Data
(60) Provisional application No. 63/251,434, filed on Oct. 1, 2021.

(51) Int. Cl. G03F 7/00; C09D 11/101; C12N 5/00

(52) U.S. Cl. CPC G03F 7/7005; C09D 11/101; C12N 5/0068; G03F 7/70291; G03F 7/70416; C12N 2533/52

(57) ABSTRACT

A method of forming a bioactive pattern on a substrate can include contacting a substrate comprising a prepolymer ink coated thereon with abeam pen lithography pen array. The prepolymer ink can include a photoinitiator, an acrylate, and a thiol-modified or acrylate-modified functional binding molecule. The method can further include irradiating the beam pen lithography pen array to transmit the radiation through the pens and out the exposed tip to controllably irradiate the prepolymer ink to initiate selectively photopolymerization of the prepolymer ink and form a pattern of thiol-functionalized cross-linked polymer printed indicia on the substrate; and exposing the pattern of the thiol-functionalized cross-linked polymer printed indicia in a biomolecule containing solution under conditions sufficient to bind the biomolecule to the thiol-functionalized cross-linked polymer printed indicia to form the bioactive pattern.

### STATEMENT OF GOVERNMENT SUPPORT

This invention was made with government support under grant numbers FA9550-16-1-0150 and FA9550-18-1-0493 awarded by the Air Force Office of Scientific Research (AFOSR) and under grant number 2032180 awarded by the National Science Foundation (NSF). The government has certain rights in the invention.

### BACKGROUND

It is important in certain electronics, materials chemistry, and biological applications to be able to control and synthesis of functional features with sub-micron to nanometer-scale precision. For example, surface engineering using polymeric materials has proved successful for the fabrication of 3D functional microrobots and on-chip thermoresponsive actuators. In addition, micropatterned extracellular matrix (ECM) proteins can be used to regulate the in vitro cellular microenvironment and, therefore, spatiotemporally tune cell behavior. In particular, the geometry of patterned proteins has been used to modulate and control cell viability and growth, stem cell differentiation, and cell orientation and migration.⁵ Also, advances in patterning technologies have enabled the precise positioning of cell clusters and bioactive materials, opening new avenues for high-throughput drug screening and bioactive detector and live-cell-based sensor fabrication.

A variety of bioprinting techniques, including electron-beam lithography, nanoimprint printing, photolithography, and scanning probe lithography, have been used to precisely arrange live cells and biomaterials in an arbitrary manner on surfaces. For instance, Harnett et al. fabricated 300-nm wide antibody lines using e-beam irradiation and demonstrated the successful attachment of avidin-coated beads or fluorescent anti-biotin IgG onto them. However, conventional bioprinting methods are inherently limited in their ability to be used to produce nano-/micro-scale patterns. For example, nanoimprint printing and photolithography have proven to be powerful tools for the generation of bioactive protein arrays with micrometer to nanometer fidelity; however, pre-formed patterns can only be created and duplicated (i.e., stamps or photomasks). In comparison, photoprinting systems based on digital light processing (DLP) afford one the flexibility to create arbitrary functional micropatterns but encounters resolution limitations (generally between 20 to 100 microns) when constructing nanoscale bioactive patterns.

In the past decade, cantilever-free scanning probe techniques have been extensively explored in micropatterning applications in biomedicine. Polymer pen lithography (PPL) has been utilized to produce fibronectin microarray templates over a large area (millimeter scale) for controlled cell attachment and single cell studies. Furthermore, a photo-controlled scanning probe system (i.e., beam pen lithography (BPL)) was developed to precisely direct photons to arbitrary interfaces via arrays of millions of pyramid-shaped polymer probes. By using arrays of gold-coated tips with nanoscopic apertures at their apexes, BPL enables control over photochemical reactions beyond the diffraction limit of light. Distinct patterns have been produced simultaneously over a large working area with massively individually addressable tips in a BPL array. As such, BPL is a powerful tool for controlling photochemical composition with nanometer resolution, thereby opening avenues for surface modification, materials discovery and biological study.

### SUMMARY

In accordance with the disclosure, a cantilever-free scanning probe lithography (CF-SPL)-based method for the rapid polymerization of nanoscale features in 2.5D on a surface via crosslinking and thiol-acrylate photoreactions can allow for the position, height, and diameter of each feature can be finely and independently tuned. With precise spatiotemporal control over the illumination pattern enabled by a piezoactuator and a digital micromirror device (DMD), beam pen lithography (BPL) allows for the photo-crosslinking of polymers into ultrahigh resolution features over centimeter-scale areas using massively parallel arrays of individually addressable pens that guide and focus light onto the surface with sub-diffraction resolution. The photoinduced crosslinking reaction of the ink material, which is composed of a photoinitator, a diacrylate, and a thiol-modified species, achieved approximately 80% conversion with limited UV intensity (75 mW/cm²) and exposure time (0.5 s), making it a valuable addition to the on-surface chemistry toolbox for high-speed BPL printing. It is demonstrated that such polymer patterns can be further reacted with proteins to yield high-resolution protein arrays with arbitrary protein arrangements and densities precisely controlled via local UV dosage. This platform, which combines polymer photochemistry and scanning probes, affords a new approach to making arbitrary protein microarrays in a high-throughput and high-yield manner, opening new routes in biochip synthesis, biomolecular detection and screening, and cell biology research.

A method of forming a bioactive pattern on a substrate can include contacting a substrate comprising a prepolymer ink coated thereon with a beam pen lithography pen array. The beam pen lithography pen array can have a plurality of pens extending from a common substrate, each pen having a base attached to the common substrate and an oppositely disposed tip, a blocking layer is coated on each pen and has an aperture through which the tip is exposed. The prepolymer ink can include a photoinitiator, an acrylate, and a thiol-modified or acrylate-modified functional binding molecule. The method can further include irradiating the beam pen lithography pen array to transmit the radiation through the pens and out the exposed tip to controllably irradiate the prepolymer ink to initiate selectively photopolymerization of the prepolymer ink and form a pattern of thiol-functionalized cross-linked polymer printed indicia on the substrate; and exposing the pattern of the thiol- or acrylate-functionalized cross-linked polymer printed indicia in a biomolecule containing solution under conditions sufficient to bind the biomolecule to the thiol or acrylate-functionalized cross-linked polymer printed indicia to form the bioactive pattern.

### BRIEF DESCRIPTION OF THE DRAWINGS

FIG. 1A is a schematic illustration of beam pen lithography (BPL) controlled, high-resolution printing on a surface, showing UV light passing through the apertures of the pyramid-like pens locally exposing the pre-polymer ink material, and that the photoreaction progress is precisely controlled to form arbitrary patterns.

FIG. 1B is a schematic illustration of BPL-controlled photopolymer printing and protein immobilization.

FIG. 1C is a schematic illustration of a reaction scheme of BPL-induced cross-linked network formation, showing that light emitted from the tips during BPL induces the cleavage of photoinitiator diphenyl(2,4,6-trimethylbenzoyl) phosphine oxide (TPO) to release radical species for initiating the homopolymerization of poly(ethylene glycol) diacrylate (PEGDA) and forming a cross-linked network as a scaffold; simultaneously triggering a radical-mediated thiol-acrylate coupling reaction and incorporating target binding thiolated molecules (e.g., thiol-PEG-biotin, MHA) into the polymer matrix.

FIG. 2A is an optical microscopy image of BPL-printed biotin containing cross-linked polymer patterns showing a representative area of ˜10,000 duplicates of 4×4 dot features, using 0.5 s of 90 mW/cm² UV exposure and 800 mN of applied force. The inset shows an AFM image of the polymer pattern, and the average feature diameter was measured to be 367±21 nm

FIG. 2B is a fluorescence microscopy image of a biotin-containing array treated with fluorescently labeled streptavidin (streptavidin-Cy3), showing uniform immobilization of the protein on a BPL-printed pattern.

FIG. 2C is an AFM image of BPL-printed 10×10 cross-linked polymer features, using dwell times (y-axis) of 0.2, 0.4, 0.6, 0.8, 1.2, 1.4, 1.6, 1.8, 2.0 s and printing forces of 200, 300, 400, 500, 600, 700, 800, 900, 1000, 1100, 1200 mN.

FIG. 2D is a graph showing the evolution of the feature height and full width at half maximum (FWHM) of the BPL-printed polymer as a function of exposure time at the printing force of 1200 mN. (The corresponding height profile measured by AFM is shown in FIG. 11).

FIG. 3A is a graph showing the evolution of acrylate infrared absorption peaks as a function of irradiation time as measured with attenuated total reflectance (micro-FT-IR, Bruker). The PEGDA photopolymerization kinetics was evaluated by monitoring the decrease in the area of the acrylate peak at 1,408 cm⁻¹

FIG. 3B shows plots of acrylate conversion versus time for PEGDA polymerization with thiol-PEG-biotin (red) and simple PEGDA polymerization (black). The ink material was spin-coated on a gold substrate and irradiated with 405 nm light with exposure times of 0.5, 1, 2, 4, or 5 s. The samples were composed of TPO, PEGDA, and thiol-PEG-biotin (0.2 g/L, 21.2 g/L, and 1 g/L in DMF, respectively).

FIG. 4A is a fluorescence image of a fluorescently labeled streptavidin (Streptavidin-Cy3) array printed on the pattern prepared using BPL with a constant force of 900 mN and illumination times of 0.2, 0.4, 0.6, 0.8, 1.0, 1.2, 1.4, 1.6, 1.8, and 2.0 s (increasing from right to left). The inset shows a 10×10 gradient array printed by a single pen.

FIG. 4B is a fluorescence intensity profile of the features printed with different exposure times indicated by the white line in FIG. 4A.

FIG. 4C is a fluorescence microscopy image of MHA-containing polymer pattern treated with fluorescently labeled fibronectin (rhodamine fibronectin), showing the attachment of fibronectin on the BPL-printed micropattern.

FIG. 5A is a fluorescence microscopy image of fluorescently labeled streptavidin (streptavidin-Cy3) arrays of varied shapes (e.g., triangle, square, hexagon) printed by BPL using an applied force of 900 mN and a UV exposure time of 0.8 s. The 50×50 μm protein patterns consisted of 750±50 nm dot features with spacing of 2.5 μm. The inset shows a square pattern formed by dot features printed using a single tip.

FIG. 5B is a fluorescence microscopy image of “QR code” patterns generated after treatment with fluorescently labeled streptavidin. The inset shows the optical microscopy image of “QR code” printed by a single tip.

FIG. 5C is an AFM image of the polymer pattern printed by an individual tip showing a continuous pattern formed by a pixel size of 830±70 nm.

FIG. 6 is a scanning electron microscope (SEM) image of a gold-coated BPL array with 25 μm spacing between 16 μm×16 μm pyramid-shaped pens with ˜800-nm apertures at their tips. With appropriate etching conditions, BPL arrays with different sized apertures, ranging from 300 to 1,500 nm, can be fabricated.

FIG. 7 is a graph showing the size distribution of the dot features of PEGDA polymer (the one used in FIG. 1a). AFM indicates that the average size of the printed features was 367±21 nm.

FIG. 8 is a graph showing the relationship between fluorescence intensity and initial concentration of thiol-PEG-biotin in DMF (0.02 mM, 0.12 mM, 0.62 mM, or 3.12 mM). The amount of attached streptavidin, indicated by the measured fluorescence intensity, increases as a function of the amount of the thiol-PEG-biotin incorporated within the polymer network. In the control group, biotin-PEG without a thiol modification was used, and fluorescence was not observed after streptavidin treatment.

FIG. 9A is an optical and fluorescent image of (top) of the control group where biotin without a thiol modification was added into the PEGDA photopolymer. After incubation in PBS solvent overnight, streptavidin attachment was not observed.

FIG. 9B is an optical and fluorescence image of a PEGDA polymer pattern with an identical amount of biotin-PEG-thiol. After incubation in PBS solvent overnight, streptavidin attached to the polymer pattern and fluorescence was observed.

FIG. 10A is an optical microscopic image of BPL-printed gradient polymer features, using dwell times (y-axis) of 0.2, 0.4, 0.6, 0.8, 1.2, 1.4, 1.6, 1.8, and 2.0 s and printing forces of 200, 300, 400, 500, 600, 700, 800, 900, 1,000, 1,100, and 1,200 mN.

FIG. 10B is a zoomed in portion of the optical microscope image of FIG. 10A annotated to show the patterning exposure time and applied force to produce the gradient features.

FIG. 11 is a graph showing the height profile of the polymer features printed using exposure times ranging from 0.2 to 2.0 s and a constant force of 1,200 mN (corresponding to FIG. 2d). The dimensions of the features in the gradient pattern were evaluated by measuring their heights and the FWHMs of their feature profiles.

FIG. 12 is a graph showing the evolution of acrylate IR absorption peaks during BPL printing. Measured with attenuated total reflectance (micro-FT-IR, Bruker), the kinetics of the thiol-acrylate reaction and PEGDA photopolymerization were evaluated by monitoring the corresponding decrease in IR peak area at 1,408 cm⁻¹ (the peak corresponding to the in-plane scissoring vibration of the acrylate groups). The acrylate peak was integrated from 1,400 cm⁻¹ to 1,430 cm⁻¹. The polymer pattern was irradiated with a UV intensity of 72 mW/cm² UV exposure (same condition as was used during BPL).

FIGS. 13A and 13B show preliminary cell attachment (NIH 3T3) on photo-printed fibronectin arrays. NIH 3T3 fibroblast cells were immobilized onto the photo-printed fibronectin micropatterns (square of 40×40 μm). The NIH 3T3 cells grew along the polymer features; non-specific binding and the attachment of multiple cells on single printed features were observed.

FIG. 14 is an optical microscopy image of “QR code” patterns generated by BPL using an applied force of 900 mN and an exposure time of 0.8 s.

FIGS. 15A and 15B show a DMD (20×, each pixel is 1.25 μm)-printed polymer patterns composed of multiple proteins (streptavidin and fibronectin). MHA-containing PEGDA polymer was first printed by UV exposure. After removing the unreacted ink, biotin thiol-containing PEGDA polymer was spin-coated on the array and photocured at the same patterning area. The resulted polymer pattern was treated with Cy3-labeled streptavidin and rhodamine-labeled fibronectin subsequently, giving arrays composed of multiple arbitrary proteins.

### DETAILED DESCRIPTION

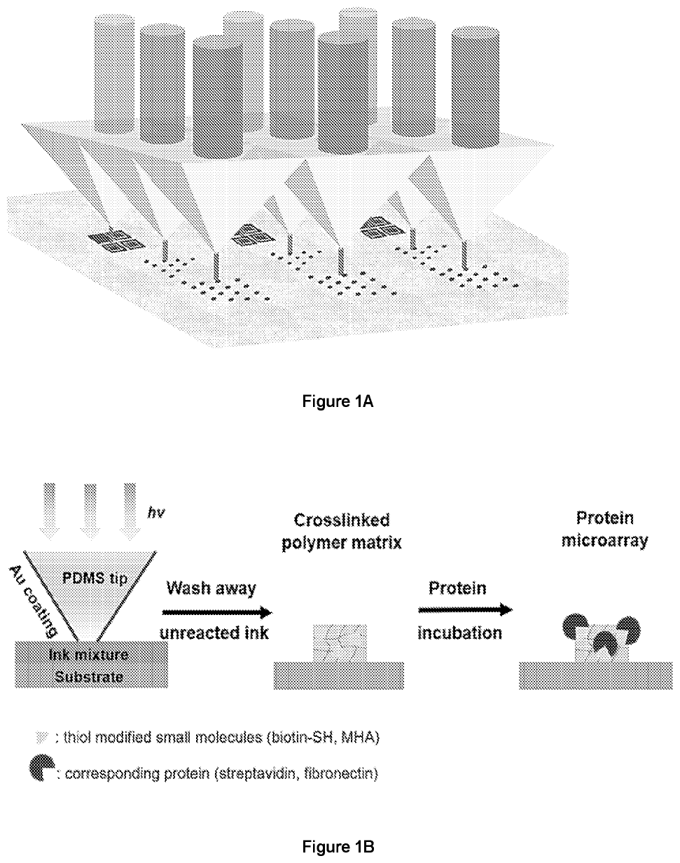

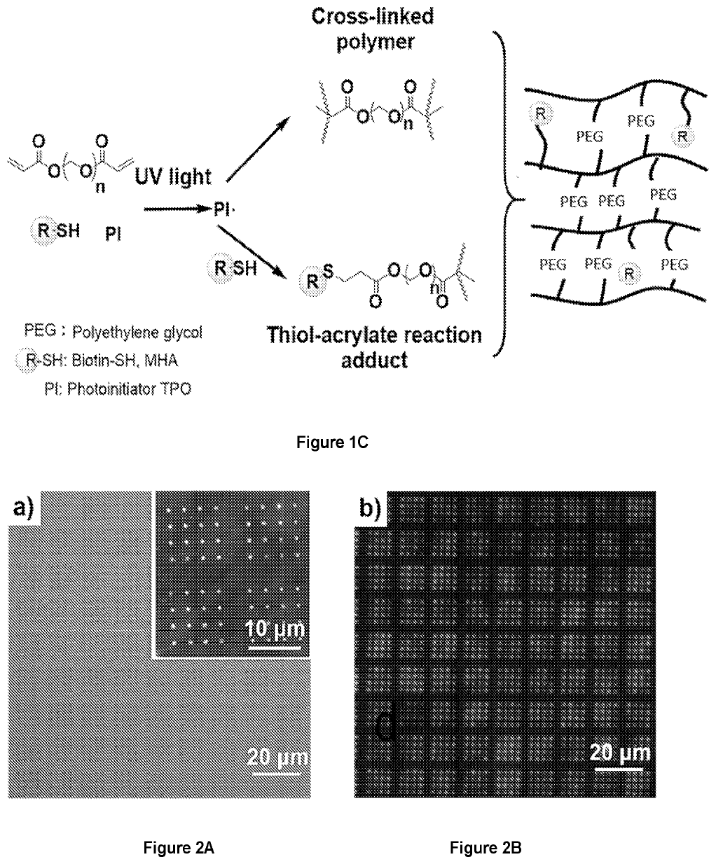

In processes of the disclosure beam pen lithography (BPL) is combined with cross-linking photopolymerization and thiol-acrylate coupling chemistry and can be used to print protein microarrays with ultrahigh resolution. Contemporary strategies for producing functional bioactive microarrays (i.e., linear polymer synthesis) yield highly controllable polymer growth; however, relatively long reaction times (generally over 10 minutes) are required and chain-length limitations, due to the deactivation or embedding of the initiating ends, exist. In contrast, the BPL-based photoinduced cross-linking reaction of multifunctional acrylates proceeds more rapidly (in a few seconds) and thiol-modified target molecules (for example, biotin and 6-mercaptohexanoic acid, MHA) can be incorporated simultaneously into the cross-linked network via thiol-acrylate coupling reactions (FIG. 1). Furthermore, high-resolution protein microarrays (for example, of streptavidin and/or fibronectin) can be achieved subsequently via streptavidin-biotin and MHA-fibronectin coupling reactions. By precisely controlling the radiation dosage via the modulation of exposure time (seconds) and contact force (mili-newtons), methods of the disclosure affords exquisite control over the photoreaction conditions on the substrate, thus making the photopolymerization and thiol-acrylate reactions highly tunable. This nanolithographic method enables the fabrication of nanoscale functional polymer features (resolution <300 nm) over large printing areas (3.8 mm×5.1 mm), making these protein micropatterns adaptable for numerous applications. The BPL-printed bioactive polymers exhibit high protein binding affinity, and the amount of immobilized protein can be controlled based on photopolymer growth. Lastly, the addressability of the individual BPL pens allows the generation of arbitrary arrangements of 2D features while sub-diffraction resolution is maintained.

Methods of the disclosure can include contacting a substrate comprising a prepolymer ink coated thereon with a beam pen lithography pen array and irradiating the beam pen lithography pen array to selectively and controllable irradiate the prepolymer ink to initiate photopolymerization of the prepolymer ink in the exposed regions and form a pattern of thiol- or acrylate-functionalized cross-linked polymer printed indicia on the substrate. The printed indicia can have feature sizes on the nanoscale. For example, the printed indicia can have an effective average diameter of less than 500 nm. Controllable and selective irradiation of the prepolymer ink solution is achieved by virtue of using the beam pen lithography system in which a pen array having a blocking layer disposed on each of the pens with an aperture in the blocking layer through which the tip of the pens is exposed. Irradiation of the beam pen lithography pen array results in transmission of the radiation through the pens and out the exposed tip. Bioactive patterns can then be formed by exposing the pattern of the thiol- or acrylate-functionalized cross-linked polymer printed indicia in a biomolecule containing solution under conditions sufficient to bind the biomolecule to the thiol- or acrylate-functionalized cross-linked polymer printed indicia to form the bioactive pattern.

In any of the methods disclosed herein, the beam pen array can be as known in the art, such as for example described in U.S. Pat. No. 9,021,611, the disclosure of which is incorporated herein by reference in its entirety. In general, beam pen lithography pen arrays include a plurality of pens extending from a common substrate. Each pen has a base fixed to the common substrate and an oppositely disposed tip. Each pen is formed of a deformable material and coated with a blocking layer. The blocking layer includes an aperture through which the tip of the pen is exposed. Radiation is capable of being controllable delivered to a substrate through the pens and out the exposed tip. Near field optical effects can be generated when the pen array is brought into proximity with the substrate and deformation of the pens when contact is made with the substrate can provide a change in the size of near-field optical effects. This can allow for precise control over the photopolymerization of the printed indicia in methods of the disclosure. Further control of the irradiation exposure is further tuned through the dwell time and/or contact pressure of the pens on the substrate.

For example, methods of the disclosure can include contacting the substrate with a printing force of about 1 mN to about 10,000 mN, about 100 mN to about 3000 mN, about 4000 mN to about 7000 mN, or about 600 mN to about 1200 mN. The dwell time can be about 0.05 seconds to about 100 seconds, about 0.2 seconds to about 5 seconds or about 0.5 seconds to about 100 seconds, about 5 seconds to about 100 seconds, about 30 seconds to about 75 seconds, or about 10 seconds to about 45 seconds. The light intensity can be 405 nm, 72 mW/cm²

The methods of the disclosure can further include repeatedly contacting the substrate with the pens of the beam pen lithography pen array and selectively and controllable irradiating the prepolymer ink to form a pattern of printed indicia across the substrate.

The prepolymer ink includes a photoinitiator, an acrylate, such as methacrylate, and a thiol-modified functional biomolecule.

The photoinitiator can be one or more of diphenyl(2,4,6-trimethylbenzoyl) phosphine oxide (TPO), lithium phenyl-2,4,6-trimethylbenzoylphosphinate (LAP), phenyl bis (2,4,6-trimethylbenzoyl) phosphine oxide (BAPO), camphorquinone (CQ), ethyl-dimethylamino benzoate (EDAB), Omnirad TPO-L, Omnirad 819, Irgacure 2959, Irgacure 651, Irgacure 184, Darocur 1173, Irgacure 819, Eosin-Y, Riboflavin, Camphorquinone and Isopropylthioxanthone (ITX).

The acrylate can be polyacrylate, polymethacrylate and poly(ethylene glycol) diacrylate (PEGDA) based polymers, such as PEGDA (Mn 4000), poly(ethylene glycol) methacrylate, and/or poly(methyl methacrylate).

The thiol-modified or acrylate-modified functional binding molecule can be selected depending on the desired biomolecule to be bound or immobilized on the pattern. For example, the thiol-modified and/or acrylate-modified functional binding molecule can be one or more of thiol-PEG-biotin, 6-mercaptohexanoic acid (MHA), thiol-PEG-OH, thiol-PEG-COOH, thiol-PEG-NH2, thiol-PEG-Azide, acrylate-PEG-biotin, acrylate-PEG-OH, acrylate-PEG-COOH, acrylate-PEG-NH2, acrylate-PEG-Azide, peptides with thiol or acrylate functionality, and thiol or acrylate/methacrylate modified nucleotides.

Various biomolecules can be patterned using methods of the disclosure. For example, the biomolecule can be or include one or more of cells, proteins, lipids, antibodies, peptides, DNA, and RNA.

The method can include irradiating the polymer with light. For example, the light can have a wavelength of about 365 nm to about 530 nm. In some embodiments, the light can be UV light. The UV light can have a wavelength of about 365 nm to about 405 nm, for example. Controlled and selective irradiation of the pens of the beam pen lithography pen array can be achieved using a digital micromirror to emit the light.

The method can further include washing the substrate having the pattern of thiol-functionalized cross-linked polymer printed indicia before immersing in the biomolecule containing solution. This can aid in removing any unreacted ink. The washing can be done in any one or more of acetone, ethanol, methanol, isopropanol, and water.

Various substrates can be used. For example, the substrate can be a silicon wafer, glass, fused silica, or quartz. For example, the substrate can be SiO₂ or an acrylate or thiol modified SiO₂. Acrylate or thiol modified other substrates, such as glass and quartz may be suitable as well. The substrate can be coated in gold prior to coating in the prepolymer ink.

The prepolymer ink can be coated on the substrate using any known methods. For example, the prepolymer ink can be spin-coated or spray-coated onto the substrate. The prepolymer ink can be deposited to a thickness of about 20 nm to about 150 nm. The spin rate of the spin-coating method can be used for example to vary the deposition thickness. The spin-coating can be performed at a range of about 500 to about 2000 rpm.

The process can include forming a pattern of two or more biomolecules. In such embodiments, the process can include subsequently coating the substrate with a subsequent prepolymer ink after forming a first (or preceding) pattern of printed indicia. Each prepolymer ink coated on the substrate can have a thiol-modified binding molecule that is adapted to bind different biomolecules. After the desired number of different patterns of thiol-functionalized cross-linked polymer printed indicia are formed, the substrate can be exposed to a solution containing the desired biomolecules to bind the respective molecules to the printed indicia having the corresponding thiol-modified binding molecule.

A process in accordance with the disclosure was performed to directly print pre-polymer material on gold-coated substrates to prepare templates for high-resolution protein microarrays. The ink, which included the radical photoinitiator diphenyl(2,4,6-trimethylbenzoyl) phosphine oxide (TPO), poly(ethylene glycol) diacrylate (PEGDA), and thiol-modified functional binding molecules (i.e., thiol-PEG-biotin or 6-mercaptohexanoic acid (MHA)), was photopatterned using a BPL pen array with UV light (A 405 nm) emitted from a digital micromirror device (DMD) (FIG. 1a). The DMD enables pen actuation to control surface irradiation, so that distinct nanoscale features at the subwavelength scale and complicated ultrahigh-resolution microarrays can be fabricated. Upon UV exposure, the photopolymerization of acrylate and the thiol-acrylate reaction reached ultimate conversion rapidly within a few seconds in the presence of TPO (FIGS. 1b and 1c). The functional binding molecules were covalently incorporated into the PEGDA cross-linked polymer network via a thiol-acrylate addition reaction during printing; then a specific protein binding treatment was used to obtain the desired protein patterns. Given the well-explored acrylate/methacrylate photopolymerization process and an extensive library of thiol-functionalized molecules can allow process of the disclose using cross-linked photopolymer printing to have potential use beyond the synthesis of protein microarrays and biochips.

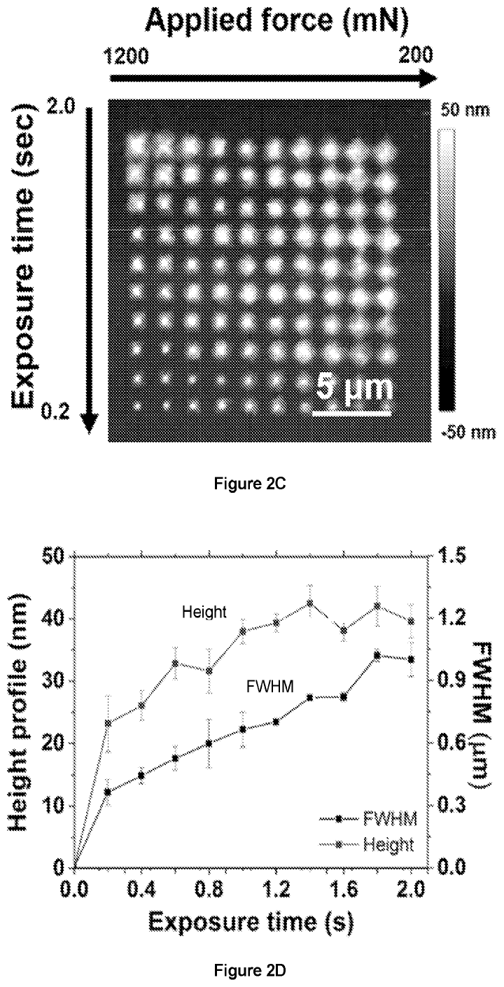

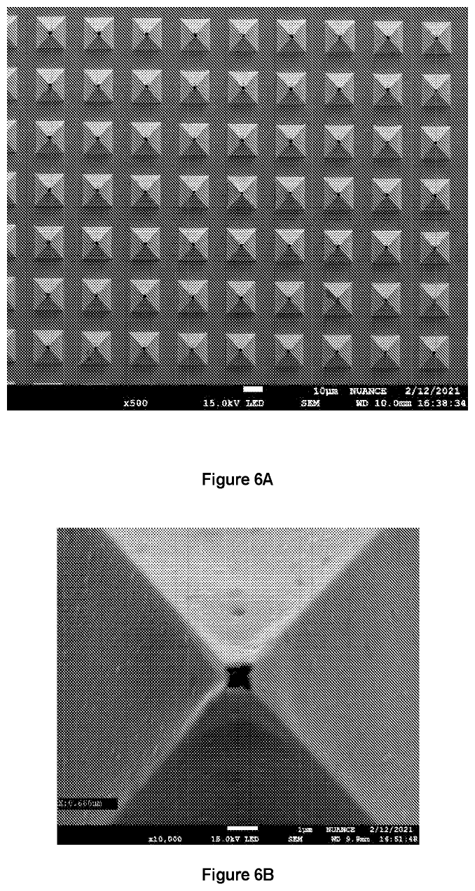

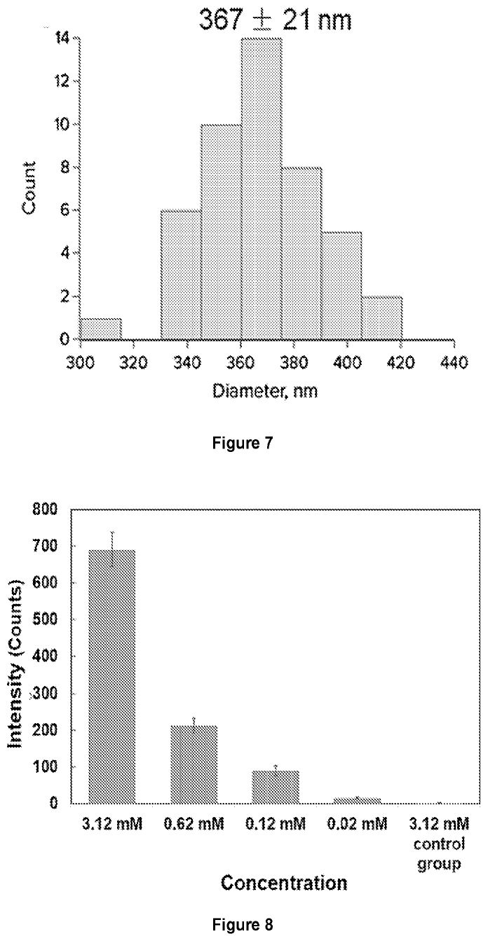

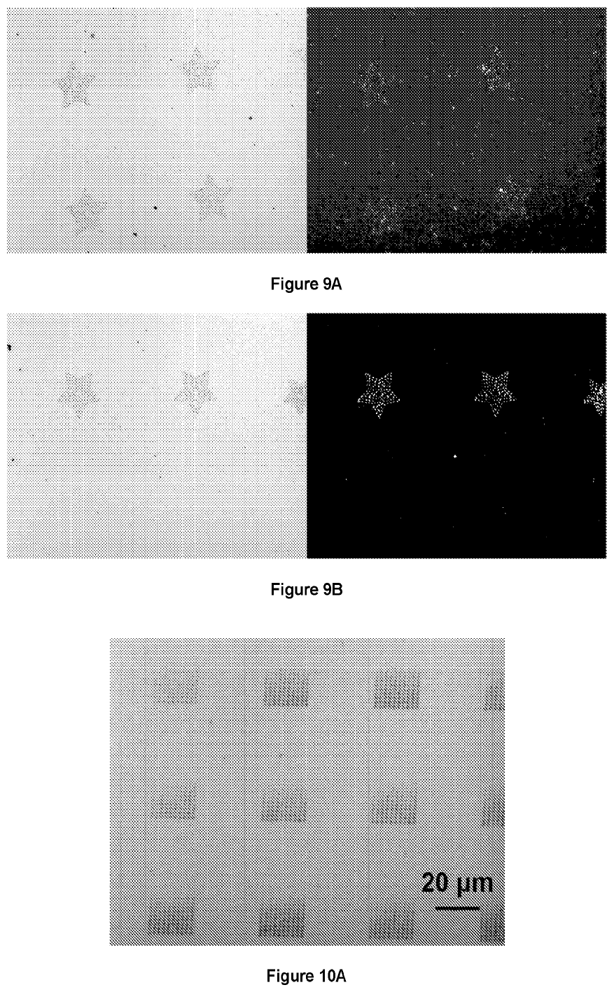

In an example, the process generated ultra-high resolution protein patterns using a TPO, PEGDA, and a thiol-PEG-biotin photopolymerization system with subsequent attachment of fluorescently labeled streptavidin. Due to its high stability and binding specificity, thiol-PEG-biotin was implemented as the target-binding species during lithographic printing. Before BPL printing, the gold surface was passivated using poly (ethylene glycol) methyl ether thiol (PEG) to minimize the non-specific binding of proteins (or cells) to the non-patterned areas. A pre-polymer ink composed of TPO, PEGDA, and thiol-PEG-biotin (0.2 g/L, 21.2 g/L, and 1 g/L, respectively) was dissolved in NN-dimethylformamide (DMF) and then spin-coated onto the PEG-treated gold surface to form a uniform pre-polymer layer. The BPL pen array (25 μm spacing between pens and 16 μm×16 μm pyramidal pens with ˜800 nm apertures at the apex, FIG. 6) was prepared and mounted on a scanning probe system (TERA-fab® E-series), equipped with hardware and software that allows complete control over the patterning process (i.e., contact force (mN), exposure time, light intensity, and feature spacing). Illumination via BPL initiates the photo cross-linking of the PEGDA by repeatedly bringing the pen arrays into contact with the ink material on the surface (405 nm UV light, 90 mW/cm², dwell times: 0.2 to 3 s, printing force: 200 to 1,500 mN). After BPL printing, the surfaces were subsequently washed with acetone, ethanol, and Nanopure water to remove unreacted ink. When an exposure time of 0.5 s was used, uniform 4×4 dot features (average diameter of 367±21 nm) were patterned on the TPO, PEGDA, and thiol-PEG-biotin mixture. (FIG. 2a and FIG. 7). This biotin/polymer microarray was incubated in a PBS solution of fluorescently labeled streptavidin (20 μg/mL, 30 minutes), and the resultant protein pattern was evaluated using confocal microscopy (FIG. 2b). As predicted, the amount of attached streptavidin increased as more thiol-PEG-biotin was incorporated within the polymer network (as the thiol-PEG-biotin concentration in DMF increases from 0.02 mM to 3.12 mM), as determined using fluorescence microscopy (FIG. 8). In order to prove that thiol-coupling occurred and that the thiol-PEG-biotin was not simply adhering/embedding within the polymer network, a control experiment was conducted using PEG-biotin (no thiol modification). Since this molecule does not have a thiol group, it should not couple to the acrylate, and indeed, after incubation of the substrate in the dye-labeled streptavidin, fluorescence signal was not detected (FIG. 9), indicating the thiol-acrylate click reaction is not a trivial in the BPL controlled protein printing.

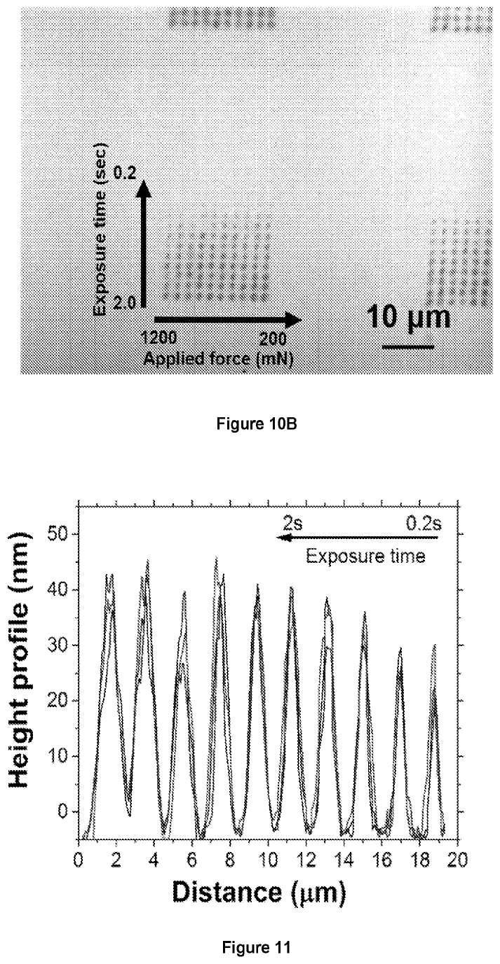

The polymerization kinetics and final reaction yield of the TPO-initiated photo reactions (i.e., the thiol-acrylate radical reaction and the chain growth propagation of the cross-linked polymer) are greatly influenced by the parameters of the UV irradiation. In methods of the disclosure, the UV exposure dose and pen-to-surface distance can be controlled by changing the dwell time and applied force, respectively, to prepare polymer features with highly tunable morphologies. BPL-patterned gradients of features (10×10) consisting of TPO, PEGDA, and thiol-PEG-biotin were synthesized using different dwell times (0.2 to 2 s) and printing forces (200 mN to 1,200 mN) (FIG. 2c, and optical microscopic image is shown in FIG. 10). The dimensions of the polymer features were evaluated by measuring the height and the full width at half maximum (FWHM) of the feature profiles in an AFM experiment. The data indicate that the height and FWHM increase as a function of exposure time for moderate printing forces (600 mN to 1,200 mN) (FIG. 2d); in this regime, the pens are close enough to the substrate so that light scattering and attenuation are avoided, allowing for tight control over the dimensions of the printed features. Indeed, nanoscale polymer features as small as 380±50 nm were fabricated. In contrast, when low printing forces (i.e., 200 to 600 mN) were applied, BPL could not be used to fabricate tunable nanoscale features by controlling the UV exposure.

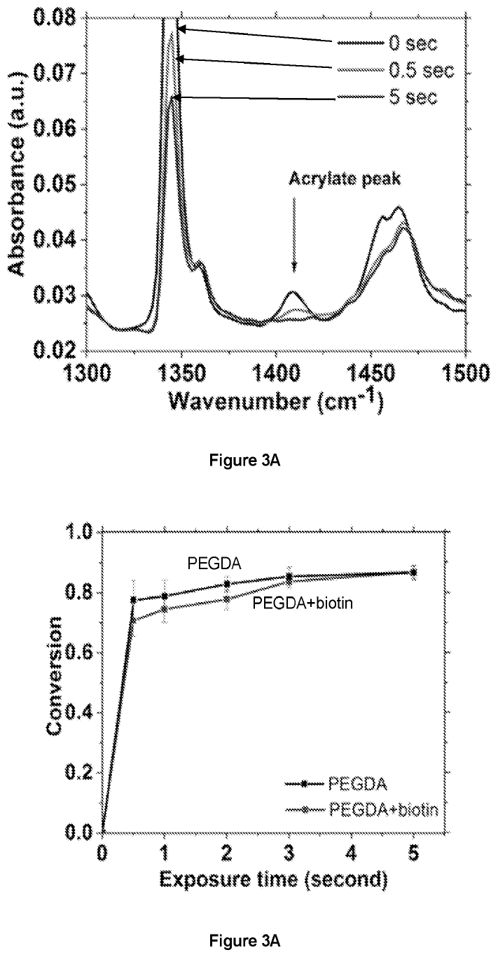

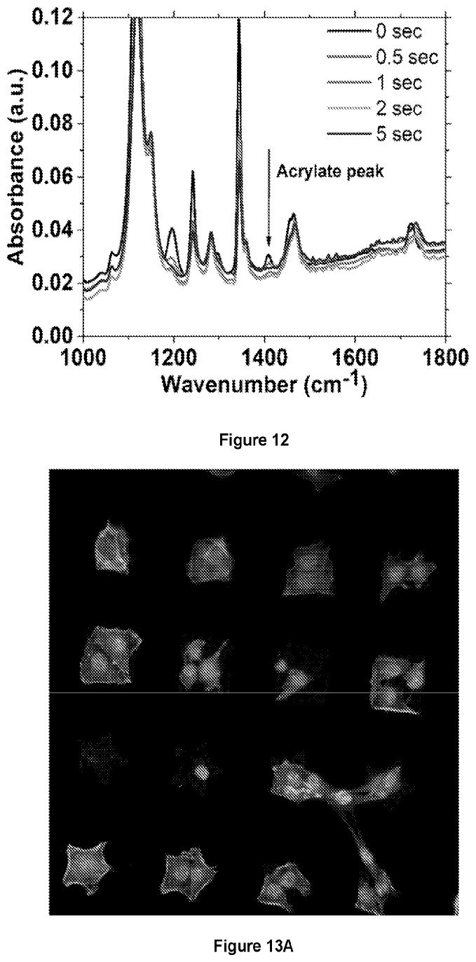

To evaluate photo chemical reactions of the ink material during printing, the photopolymerization kinetics of PEGDA was measured by monitoring the acrylate consumption using attenuated total reflection (ATR) Fourier transform infrared (FT-IR) spectroscopy (FIG. 3a). Upon 0.5, 1, 2, 3, or 5 s of UV exposure (72 mW/cm², bulk irradiation, BPL not performed but same exposure conditions), the acrylate peak observed at 1,408 cm⁻¹ decreased in area, indicating that the photoinduced cross-linking reaction approaches maximal conversion (approximately 80%) with only 0.5 s of 405 nm light irradiation (FIG. 3b, FIG. 12). The robust cross-linking reaction of PEGDA in the solid phase proceeds with rapid kinetics (even comparable to commercially available photoresists), making it suitable for high-throughput and high-yield BPL. Interestingly, the photopolymerization kinetics was not significantly affected by the thiol additive (i.e., thiol-PEG-biotin), consistent with previous thiol-acrylate photopolymerizations reported in the literature.

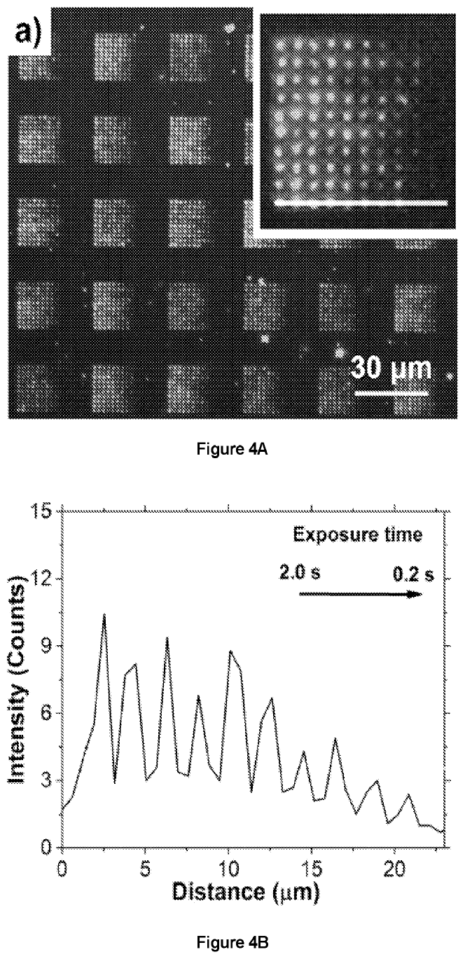

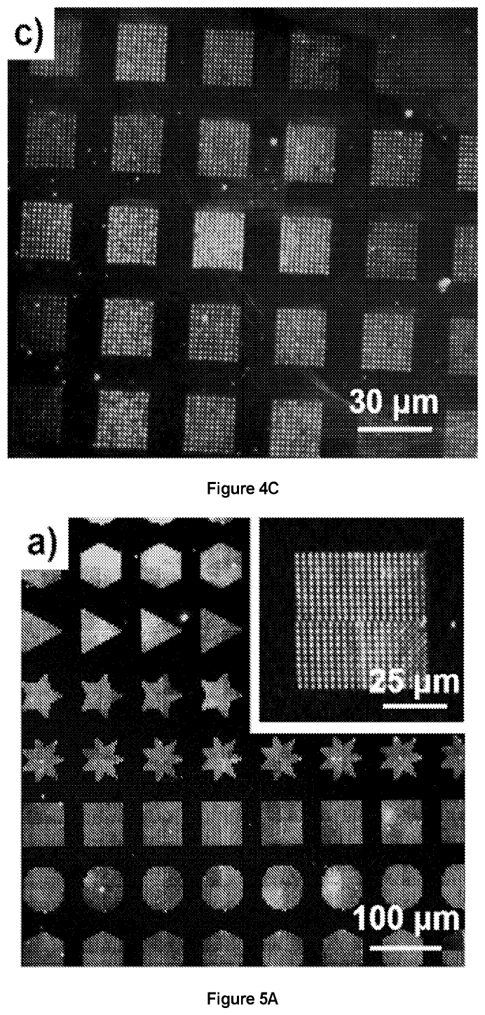

Methods of the disclosure can be used to spatiotemporally control not only the morphologies of the features composed of polymers and thiolated moieties, but also the attachment of the corresponding binding proteins through the tuning of the UV exposure conditions. Using an ink material composed of TPO, PEGDA, and thiol-PEG-biotin (0.2 g/L, 21.2 g/mL, and 1 g/mL in DMF, respectively), 10×10 gradient polymer features were printed times ranging from 0.2 s to 2 s (FIG. 4a). According to morphology control experiment (FIG. 2c), a moderate printing force of 900 mN was applied to acquire efficient control over the printed feature. The normalized fluorescence intensity, calculated as the peak fluorescence divided by the baseline fluorescence, was used to quantify the amount of protein attachment. After sufficient solvent wash and fluorescent protein treatment, a gradient of streptavidin patterns was obtained with normalized fluorescence intensity increasing from 2.4±0.2 to 10.0±0.7 with increasing UV exposure time (FIG. 4b); the normalized fluorescence intensity, calculated as the peak fluorescence divided by the baseline fluorescence, was used to understand the amount of protein attachment. These data indicate that the methods of the disclosure can be used to precisely control the amount of immobilized protein within the printed features. This property can make the methods of the disclosure suitable for applications in functional biochips synthesis, antibody/peptide detection and screening, and cell biological studies.

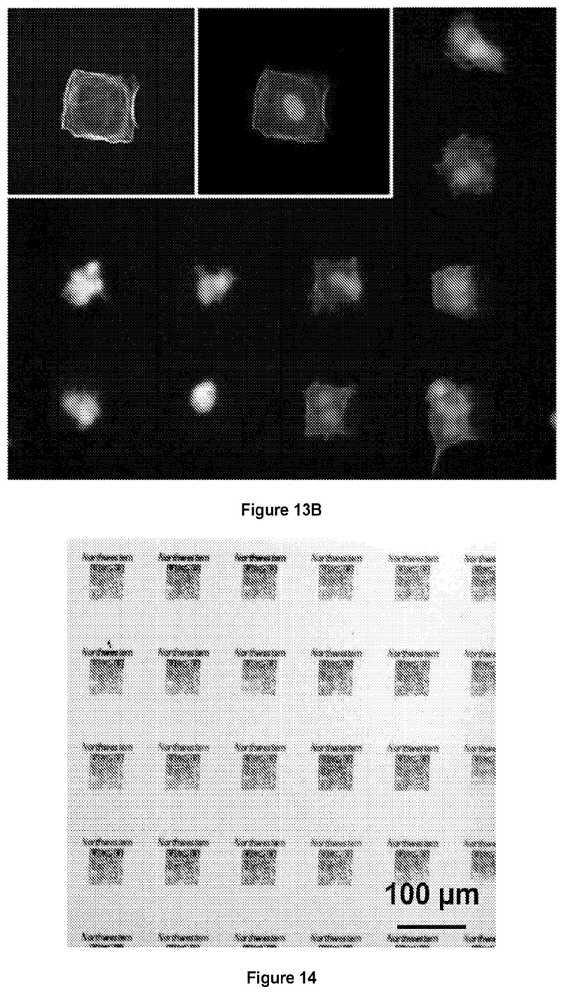

In addition, besides biotin/streptavidin arrays, other types of protein nanoscale patterns were also achieved by using the appropriate thiol-binding species in the methods of the disclosure. For example, rhodamine-labeled fibronectin was immobilized on BPL-printed PEGDA/6-mercaptohexanoic acid (MHA) polymer patterns. First, TPO, PEGDA, and MHA (0.2 g/L, 21.2 g/L, and 0.8 g/L, respectively) were mixed as a pre-polymer ink and spin-coated on a gold substrate before BPL printing (force 900 mN, exposure time 0.8 s). After removing the unreacted ink and incubating with fluorescently tagged fibronectin, high-resolution fibronectin features with an average diameter of 810±40 nm were generated and analyzed using confocal microscopy (FIG. 4c). Furthermore, the cell immobilization was attempted by attaching NIH 3T3 mouse embryonic fibroblast cells were immobilized onto these fibronectin micro patterns (40×40 μm square) (FIG. 13). Results showed successful cell attachment; however, in some cases, multiple cells attached to single printed features, and non-specific binding occurred. Advantageously, methods of the disclosure could be used to control the 3D morphology of a polymer-based fibronectin pattern as well as its mechanical properties, serving as a tool for the preparation of arbitrary bioactive arrays that mimic the cellular microenvironment for the study and control of cell motility, differentiation, and organization. This can expand on previous use of polymer pen lithography, which describe how polymer pen lithography can be used to pattern fibronectin to control focal adhesions and influence stem cell fate. Furthermore, other studies point out that the implementation of PEG in biomaterials prolongs the circulation of proteins and peptides without compromising their bioactivity.

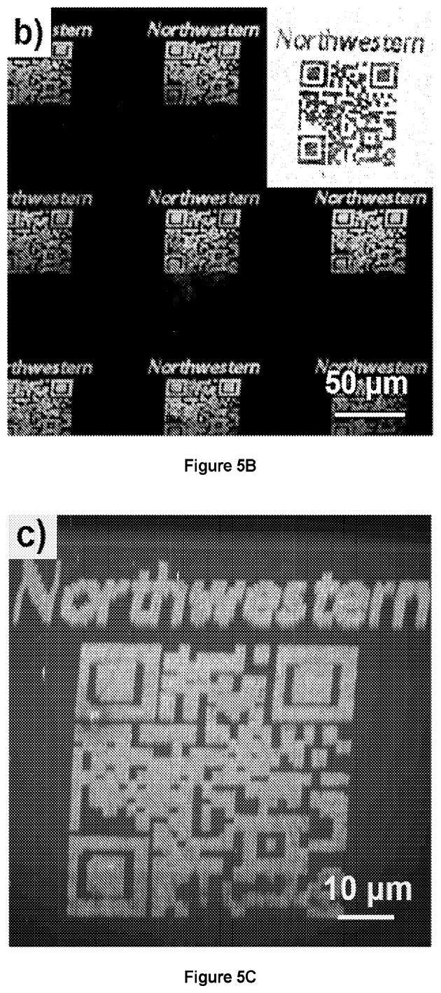

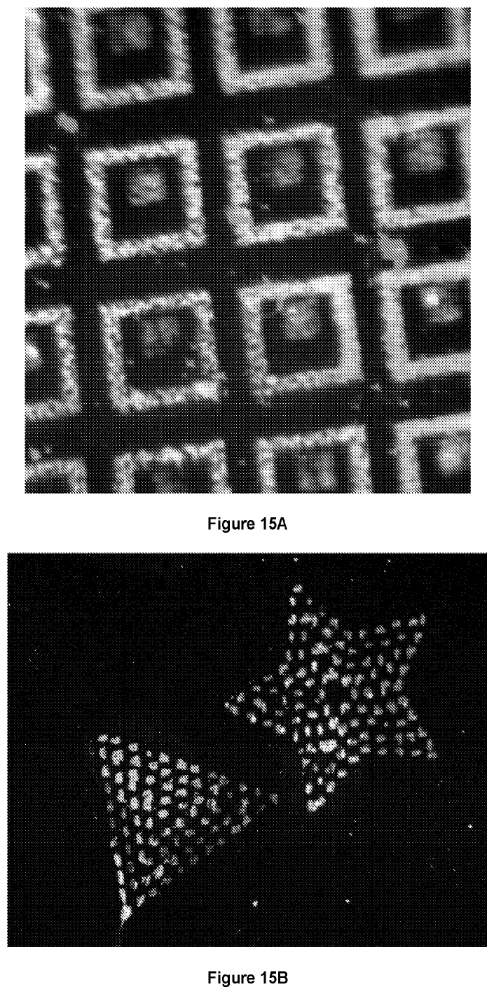

Conventional contact printing techniques, which are limited by directing cantilevers, are most often used to generate simple dot or line features in a restricted printing area. In contrast, methods of the disclosure allows one to make arbitrary patterns using massively parallel and individually addressable pens. Fluorescently tagged streptavidin microarrays with different pattern designs (e.g., triangles, squares, and hexagons, consisting of dot features with an average diameter of 750±50 nm) were fabricated using actuated BPL. A pre-polymer ink, composed of TPO, PEGDA, and thiol-PEG-biotin, was photopolymerized within 50×50 μm regions using BPL (force 900 mN, dwell time 0.8 s, exposure intensity 72 mW/cm²). After the removal of the unreacted ink, the system was incubated with Cy3-labeled streptavidin, and the protein patterns were developed (FIG. 5a). Moreover, the BPL tool's advanced software automatically synchronizes the piezo movement and UV exposure once a desired image to be patterned is uploaded. As a result, methods of the disclosure can allow one to arbitrarily generate continuous features and therefore print micropatterns with more complicated designs. For example, a QR code to the Mirkin group website (https://mirkin-group.northwestern.edu/) with a size of 50×50 μm² was printed (FIG. 5b, optical image is shown in FIG. 14). The resulted pattern was evaluated by AFM, and a continuous polymer pattern was formed with an individual pixel size of 830±70 nm (FIG. 5c). Furthermore, with the ability to immobilize different types of proteins, BPL-controlled printing also was used to fabricate arrays of multiple proteins to mimic varied biomolecular gradients or cellular arrangements. Multiplexed patterns containing both streptavidin and fibronectin were generated (FIG. 15). The DMD light control system allows spatiotemporal control in BPL for the fabrication of arbitrary protein patterns with versatile shapes at the macroscale, while also maintaining nanoscale resolution via millions of individually addressable tips.

Methods of the disclosure can be utilized to spatiotemporally control cross-linking and thiol-acrylate photopolymerization reactions to generate a series of different protein (streptavidin and/or fibronectin) micropatterns with precise control over feature position, polymer morphology, and amount of immobilized proteins. Compared to conventional linear polymer synthesis, the photoinduced cross-linking of PEGDA and thiol-ene “click” reaction proceeds rapidly; over 80% conversion with 72 mW/cm² UV light was achieved in 0.5 s, rendering it an appropriate chemistry tool for high-speed BPL printing. Furthermore, BPL allows the facile fabrication of polymer architectures with nanoscale fidelity using precise piezoactuator control and individually addressable pens. The combination of polymer chemistry with this scanning probe platform affords a new approach to making arbitrary protein arrays in a high-throughput and high-yield manner, making it a promising synthetic tool to address challenging scientific problems in the fields of materials chemistry and biological research.

The BLP controlled micropatterning of bioactive materials can enable the fabrication of arbitrary protein microarray which could be applied for protein detection, protein-biochip technology. The technique efficiently controls the protein/peptide/nucleotide immobilization on substrates and thus enhanced the fundamental understanding in biological fields such as biosensor synthesis, tissue engineering and cell biology.

This printing technique implements beam pen lithography as a powerful tool to fabrication arbitrary protein micropatterns on surfaces. Compared to conventional nanoscale printing techniques, such as imprint printing, e-beam lithography, and photolithography, BPL printing produces micropatterns with nanometer fidelity over a large surface are (4 mm×5 mm) without using a preformed pattern (i.e., imprinting patterns and photomask). Cross-linked photopolymer system is utilized due to its rapid curing kinetics, making it suitable for high-throughput BPL printing; the implementation of thiol=click photochemistry in the system enables the immobilization of varied thiol modified functional molecules, such as peptides, nucleotides and small molecules.

### EXAMPLES

Polyethylene glycol diacrylate (PEGDA, M.W. 3,400) was purchased from Thermo Fisher. Diphenyl (2,4,6 trimethylbenzoyl) phosphine oxide (TPO), PBS solution, streptavidin-Cy3, biotin (>99%, lyophilized powder), and 6-mercaptohexanoic acid (MHA, 90%) were purchased from Sigma-Aldrich. SH-PEG-Biotin, (M.W. 400) was purchased from Biochempeg. (1-Mercapto-11-undecyl) hexa(ethylene glycol) was purchased form Asemblon. Rhodamine-labeled fibronectin was purchased from Cytoskeleton. DyLight 488-conjugated streptavidin (21832) was purchased from Invitrogen. Fibronectin(FC010) was purchased from Sigma Aldrich. The N1H 3T3 cell line was purchased from ATCC. All chemicals and reagents were used as received without further purification.

### Preparation of Prepolymer Ink Material

PEGDA photopolymer containing SH-PEG-biotin: Photoinitiator TPO, PEGDA, and SH-PEG-biotin were dissolved in dimethylformamide (DMF) at concentrations of 0.2 g/L, 21.2 g/L, and 1 g/L, respectively. PEGDA photopolymer containing MHA: Photoinitiator TPO, PEGDA, and MHA were dissolved in DMF at concentrations of 0.2 g/L, 21.2 g/L, and 0.8 g/L, respectively. The sample was sonicated for 5 minutes, affording a transparent solution. The ink was filtered before it was spin-coated onto a gold substrate at a spin rate of 1,000 rpm for 90 seconds. The spin rate used ranged from 600 to 1,500 rpm, and it affected the thickness of the coated ink layer. The ink material was uniformly coated on a gold substrate and dried with room N₂ before BPL printing.

Gold substrates were made by depositing Ti/Au (thickness of 5 nm/35 nm) on Corning cover glasses (square, No. 2, W×L 18 mm×18 mm, Sigma). One mM (1-mercapto-11-undecyl) hexa(ethylene glycol) in ethanol was used to backfill the gold surface to prevent the non-specific attachment of proteins and cells (overnight incubation).

### Beam Pen Lithography

BPL pen arrays were prepared using a previously reported protocol.¹ (Some BPL arrays were provided by TERA-print, https://www.tera-print.com/). Hard polydimethylsiloxane (PDMS) arrays of pyramids with a base width of 25 μm were fabricated for BPL according to previously reported protocols. The resulting PDMS arrays were coated with 5 nm of Ti and 200 nm of Au to form an opaque layer. The tip of each probe was etched off to afford nanoscopic apertures.

The ink was spin-coated on a gold substrate. The ink material at the corners and edges was wrapped off to enable electronic contact with the BPL array and to allow for electronic alignment prior to printing. The pen array was mounted on a scanning probe system (TERA-fab E series), and the system was leveled electronically with respect to the ink-coated Au substrate. During BPL printing, the UV irradiation was controlled precisely using the DMD. Furthermore, the scanning probe instrument was equipped with an x-y tilting stage and lithography software, which allows for precise control over the dwell time and z-direction printing position during patterning.

Incubation of BPL-Printed Patterns with Fluorescent Labeled Protein

Cy3-streptavidin (25 μg/mL, PBS) was placed on a BPL-printed substrate (using PEGDA and thiol-PEG-biotin as the ink material). Specifically, the patterned features were incubated in Cy3-streptavidin for 2 h at room temperature and then washed with 1×PBS three times.

For the rhodamine-labeled fibronectin arrays, a 25 μg/mL rhodamine-labeled fibronectin solution was placed on the BPL-printed substrate (using PEGDA and MHA as ink material). The patterned features were incubated in Cy3-streptavidin for 2 h at room temperature and then washed with 1×PBS three times.

Fluorescence images were taken using a confocal microscope (Zeiss LSM 800).

### AFM Experiments

The BPL-printed polymer patterns were characterized using a Bruker Dimension Icon with a TESPA AFM tip (force constant 37 Nm⁻¹). AFM data was analyzed using the Nanoscope analysis software.

### FT-IR Experiments

The photoreaction of the pre-polymer ink on a Au surface was measured using attenuated total reflectance (Bruker LUMOS FTIR Microscope). The consumption of the acrylate moieties was monitored by measuring the IR peak area at 1,408 cm¹, which corresponds to the in-plane scissoring vibration of the acrylate groups; this peak decreased over time under irradiation. The real-time functional group conversion was calculated using the ratio of peak area to the peak area prior to the reaction.

### Culture of NIH 3T3 Cells on a Patterned Fibronectin Array

Cell-seeding substrates were made starting with 5 nm Ti/35 nm Au on fused silica. After patterning with PEGDA and MHA, the substrates were cleanly thoroughly with acetone and ethanol and dried with nitrogen. Sterilized substrates were put into six-well petri dishes and incubated in 25 μg/mL fibronectin solution at 4° C. overnight. NIH 3T3 fibroblast cells were re-suspended in serum medium (10% fetal bovine serum and 1% penicillin-streptomycin in Dulbecco's Modified Eagle Medium) and cultured with the prepared fibronectin microarrays. Briefly, 250 μL of NIH 3T3 cells (30 k) were seeded on the patterned fibronectin substrate and allowed to attach for 45 minutes. Unadhered cells were removed, and the remaining cells were rinsed once with 1×PBS. Two mL of cell media was supplemented into each well, and the entire cell culture was allowed to incubate for 6 hours under 5% CO₂ at 37° C. Such cultured cells were fixed with 4% paraformaldehyde, stained with phalloidin 594 or 488 and finally fixed with Prolong Gold antifade reagent with DAPI (Invitrogen).

### SEM Analysis

The SEM images were collected with a Hitachi SU8030 at a 15.0 kV accelerating voltage.

### REFERENCES

1. Baig, N.; Kammakakam, I.; Falath, W., Nanomaterials: a review of synthesis methods, properties, recent progress, and challenges. Mater Adv 2021, 2 (6), 1821-1871.
2. Li, Q. F.; Grojo, D.; Alloncle, A. P.; Chichkov, B.; Delaporte, P., Digital laser micro- and nanoprinting. Nanophotonics-Berlin 2019, 8 (1), 27-44.
3. Lee, H.; Fang, N. X., Micro 3D Printing Using a Digital Projector and its Application in the Study of Soft Materials Mechanics. Jove-J Vis Exp 2012, (69).
4. Liu, A. P.; Chaudhuri, O.; Parekh, S. H., New advances in probing cell-extracellular matrix interactions. Integr Biol-Uk 2017, 9 (5), 383-405.
5. Chen, L. N.; Yan, C.; Zheng, Z. J., Functional polymer surfaces for controlling cell behaviors. Mater Today 2018, 21 (1), 38-59.
6. Hynes, W. F.; Doty, N. J.; Zarembinski, T. I.; Schwartz, M. P.; Toepke, M. W.; Murphy, W. L.; Atzet, S. K.; Clark, R.; Melendez, J. A.; Cady, N. C., Micropatterning of 3D Microenvironments for Living Biosensor Applications. Biosensors (Basel) 2014, 4 (1), 28-44.
7. Agheli, H.; Malmstrom, J.; Larsson, E. M.; Textor, M.; Sutherland, D. S., Large area protein nanopatterning for biological applications. Nano Lett 2006, 6(6), 1165-1171.
8. Lindner, M.; Tresztenyak, A.; Fulop, G.; Jahr, W.; Prinz, A.; Prinz, I.; Danzl, J. G.; Schutz, G. J.; Sevcsik, E., A Fast and Simple Contact Printing Approach to Generate 2D Protein Nanopatterns. Frontiers in Chemistry 2019, 6.
9. Pla-Roca, M.; Fernandez, J. G.; Mills, C. A.; Martinez, E.; Samitier, J., Micro/nanopatterning of proteins via contact printing using high aspect ratio PMMA stamps and Nanolmprint apparatus. Langmuir 2007, 23(16), 8614-8618.
10. Turner, N. W.; Jeans, C. W.; Brain, K. R.; Allender, C. J.; Hlady, V.; Britt, D. W., From 3D to 2D: A review of the molecular imprinting of proteins. Biotechnol Progr 2006, 22 (6), 1474-1489.
11. Sorribas, H.; Padeste, C.; Tiefenauer, L., Photolithographic generation of protein micropatterns for neuron culture applications. Biomaterials 2002, 23 (3), 893-900.
12. Chai, J. A.; Wong, L. S.; Giam, L.; Mirkin, C. A., Single-molecule protein arrays enabled by scanning probe block copolymer lithography. P Natl Acad Sci USA 2011, 108 (49), 19521-19525.
13. Albisetti, E.; Carroll, K. M.; Lu, X.; Curtis, J. E.; Petti, D.; Bertacco, R.; Riedo, E., Thermochemical scanning probe lithography of protein gradients at the nanoscale. Nanotechnology 2016, 27 (31).
14. Harnett, C. K.; Satyalakshmi, K. M.; Craighead, H. G., Bioactive templates fabricated by low-energy electron beam lithography of self-assembled monolayers. Langmuir 2001, 17(1), 178-182.
15. Zhang, J. M.; Hu, Q. P.; Wang, S.; Tao, J.; Gou, M. L., Digital Light Processing Based Three-dimensional Printing for Medical Applications. Int J Bioprinting 2020, 6 (1), 12-27.
16. Eichelsdoerfer, D. J.; Liao, X.; Cabezas, M. D.; Morris, W.; Radha, B.; Brown, K. A.; Giam, L. R.; Braunschweig, A. B.; Mirkin, C. A., Large-area molecular patterning with polymer pen lithography. Nat Protoc 2013, 8 (12), 2548-2560.
17. Cabezas, M. D.; Eichelsdoerfer, D. J.; Brown, K. A.; Mrksich, M.; Mirkin, C. A., Combinatorial screening of mesenchymal stem cell adhesion and differentiation using polymer pen lithography. Methods Cell Biol 2014, 119, 261-76.
18. Huo, F.; Zheng, G.; Liao, X.; Giam, L. R.; Chai, J.; Chen, X.; Shim, W.; Mirkin, C. A., Beam pen lithography. Nat Nanotechnol 2010, 5(9), 637-40.
19. Liao, X.; Brown, K. A.; Schmucker, A. L.; Liu, G.; He, S.; Shim, W.; Mirkin, C. A., Desktop nanofabrication with massively multiplexed beam pen lithography. Nat Commun 2013, 4, 2103.
20. He, S.; Xie, Z.; Park, D. J.; Liao, X.; Brown, K. A.; Chen, P. C.; Zhou, Y.; Schatz, G. C.; Mirkin, C. A., Liquid-Phase Beam Pen Lithography. Small 2016, 12(8), 988-93.
21. Bian, S.; Zieba, S. B.; Morris, W.; Han, X.; Richter, D. C.; Brown, K. A.; Mirkin, C. A.; Braunschweig, A. B., Beam pen lithography as a new tool for spatially controlled photochemistry, and its utilization in the synthesis of multivalent glycan arrays. Chem Sci 2014, 5(5), 2023-2030.
22. Hoyle, C. E.; Bowman, C. N., Thiol-Ene Click Chemistry. Angew Chem Int Edit 2010, 49 (9), 1540-1573.
23. Lowe, A. B., Thiol-ene “click” reactions and recent applications in polymer and materials synthesis: a first update. Polym Chem-Uk 2014, 5 (17), 4820-4870.
24. Chandradoss, S. D.; Haagsma, A. C.; Lee, Y. K.; Hwang, J. H.; Nam, J. M.; Joo, C., Surface passivation for single-molecule protein studies. J Vis Exp 2014, (86).
25. Zhang, J. C.; Li, L. P.; Guo, R. L.; Zhou, H. R.; Li, Z. M.; Chen, G. X.; Zhou, Z.; Li, Q. F., Preparation of novel UV-cured methacrylate hybrid materials with high thermal stability via thiol-ene photopolymerization. J Mater Sci 2019, 54 (7), 5877-5897.
26. Cramer, N. B.; Bowman, C. N., Kinetics of thiol-ene and thiol-acrylate photopolymerizations with real-time Fourier transform infrared. J Polym Sci Pol Chem 2001, 39 (19), 3311-3319.
27. Lu, X.; Perera, T. H.; Aria, A. B.; Callahan, L. A. S., Polyethylene glycol in spinal cord injury repair: a critical review. J Exp Pharmacol 2018, 10, 37-49.
28. Wu, J.; Zhao, C.; Lin, W. F.; Hu, R. D.; Wang, Q. M.; Chen, H.; Li, L. Y.; Chen, S. F.; Zheng, J., Binding characteristics between polyethylene glycol (PEG) and proteins in aqueous solution. J Mater Chem B 2014, 2 (20), 2983-2992.

### REFERENCES

1. Eichelsdoerfer, D. J.; Liao, X.; Cabezas, M. D.; Morris, W.; Radha, B.; Brown, K. A.; Giam, L. R.; Braunschweig, A. B.; Mirkin, C. A., Large-area molecular patterning with polymer pen lithography. Nat Protoc 2013, 8 (12), 2548-2560.
2. He, S.; Xie, Z.; Park, D. J.; Liao, X.; Brown, K. A.; Chen, P. C.; Zhou, Y.; Schatz, G. C.; Mirkin, C. A., Liquid-Phase Beam Pen Lithography. Small 2016, 12(8), 988-93.
3. Liao, X.; Brown, K. A.; Schmucker, A. L.; Liu, G.; He, S.; Shim, W.; Mirkin, C. A., Desktop nanofabrication with massively multiplexed beam pen lithography. Nat Commun 2013, 4, 2103.

1. A method of forming a bioactive pattern on a substrate, comprising:
contacting a substrate comprising a prepolymer ink coated thereon with a beam pen lithography pen array, the beam pen lithography pen array comprising a plurality of pens extending from a common substrate, each pen having a base attached to the common substrate and an oppositely disposed tip, a blocking layer is coated on each pen and has an aperture through which the tip is exposed, the prepolymer ink comprising a photoinitiator, an acrylate, and a thiol-modified or acrylate-modified functional binding molecule;
irradiating the beam pen lithography pen array to transmit the radiation through the pens and out the exposed tip to controllably irradiate the prepolymer ink to initiate selectively photopolymerization of the prepolymer ink and form a pattern of thiol- or acrylate-functionalized cross-linked polymer printed indicia on the substrate; and
exposing the pattern of the thiol-functionalized cross-linked polymer printed indicia in a biomolecule containing solution under conditions sufficient to bind the biomolecule to the thiol-functionalized cross-linked polymer printed indicia to form the bioactive pattern.

2. The method of claim 1, wherein the photoinitiator is one or more of diphenyl(2,4,6-trimethylbenzoyl) phosphine oxide (TPO), lithium phenyl-2,4,6-trimethylbenzoylphosphinate (LAP), phenyl bis (2,4,6-trimethylbenzoyl) phosphine oxide (BAPO), camphorquinone (CQ), ethyl-dimethylamino benzoate (EDAB), Omnirad TPO-L, Omnirad 819, Irgacure 2959, Irgacure 651, Irgacure 184, Darocur 1173, Irgacure 819, Eosin-Y, Riboflavin, Camphorquinone and Isopropylthioxanthone (ITX).

3. The method of claim 1, wherein the acrylate is polyacrylate, polymethacrylate and poly(ethylene glycol diacrylate (PEGDA), poly(methyl methacrylate), poly(ethylene glycol) diacrylate (PEGDA) or poly(ethylene glycol) methacrylate.

4. The method of claim 1, wherein the binding molecule comprises a peptide with thiol or acrylate functionality and/or thiol- or acrylate-modified nucleotides.

5. The method of claim 1, wherein the binding molecule is thiol-modified and the thiol-modified functional binding molecule is one or more of thiol-PEG-biotin, 6-mercaptohexanoic acid (MHA), thiol-PEG-OH, thiol-PEG-COOH, thio-PEG-NH2, thiol-PEG-Azide.

6. The method of claim 1, wherein the biomolecule comprises one or more of cells, proteins, antibodies, lipids peptides, DNA, and RNA.

7. The method of claim 1, wherein the radiation has a wavelength of about 365 nm to about 503 nm.

8. The method of g claim 1, wherein the radiation comprises UV light.

9. The method of claim 8, wherein the UV light has a wavelength of about 365 nm to about 405 nm.

10. The method of claim 1, wherein the radiation is emitted from a digital micromirror device.

11. The method of claim 1, further comprising washing the thiol- or acrylate-functionalized cross-linked polymer printed indicia before immersing in the biomolecule containing solution.

12. The method of claim 11, comprising washing with one or more of acetone, ethanol, and water.

13. The method of claim 1, comprising printing the prepolymer indicial with a dwell time of about 0.05 s to about 100 s.

14. The method of claim 1, comprising applying a printing force of 1 mN to 10,000 mN when contacting the substrate with the beam pen lithography pen array.

15. (canceled)

16. The method of claim 1, wherein the substrate is coated with gold prior to coating with the prepolymer ink.

17. (canceled)

18. The method of claim 1, wherein the printed indicia have an average effective diameter of less than 300 nm.

19. The method of claim 1, comprising repeatedly contacting the substrate with the pen array and irradiating the pen array.

20. The method of claim 1, wherein the prepolymer ink is a first prepolymer ink and the thiol- or acrylate-functionalized cross-linked printed indicia are first thiol- or acrylate-functionalized cross-linked printed indicia adapted for binding a first biomolecule, the method further comprising coating the substrate having the first thiol- or acrylate-functionalized cross-linked printed indicia with a second prepolymer ink comprising a photoinitiator, an acrylate, and a thiol-modified or acrylate-modified binding molecule for binding a second biomolecule different from the first biomolecule, contacting the substrate coated with the second prepolymer ink with the beam pen lithography pen array and irradiating the beam pen lithography pen array to transmit the radiation through the pens and out the exposed tip to controllably irradiate the second prepolymer ink to initiate photopolymerization and form a pattern of second thiol- or acrylate-functionalized cross-linked polymer printed indicia on the substrate; and exposing the substrate having the pattern of first thiol- or acrylate-functionalized cross-linked polymer printed indicia and the pattern of second thiol- or acrylate-functionalized cross-linked polymer printed indicia to a biomolecule solution comprising the first and second biomolecules to bind the first and second biomolecules to the first and second thiol- or acrylate-functionalized cross-linked polymer printed indicia, respectively.

21. The method of claim 1, wherein the substrate is a silicon wafer, glass, fused silica, or quartz.

22. The method of claim 1, wherein the binding molecule is acrylate-modified and the acrylate-modified binding molecule is one or more of acrylate-PEG-biotin, acrylate-PEG-OH, acrylate-PEG-COOH, acrylate-PEG-NH2, acrylate-PEG-Azide.
